(19) United States
(12) Patent Application Publication
(10) Pub. No.: US 2020/0178572 A1
(43) Pub. Date: Jun. 11, 2020

(54) MICROWAVE PROCESSING METHODS FOR FORMULATING ORALLY INGESTIBLE COMPOSITIONS COMPRISING LIPOPHILIC ACTIVE AGENTS

(71) Applicant: PoViva Tea, LLC, Reno, NY (US)

(72) Inventors: John Docherty, Ontario (CA); Christopher Andrew Bunka, Kelowna (CA)

(21) Appl. No.: 16/623,367

(22) PCT Filed: Jun. 19, 2018
(86) PCT No.: PCT/US18/38232

§ 371 Date: Dec. 16, 2019

Related U.S. Application Data
(60) Provisional application No. 62/519,511, filed on Jun. 14, 2017.

(51) Int. Cl. A23L 2/39; A23L 2/56; A23L 29/212; A23L 29/10; A61K 45/06; A61K 47/44; A61K 47/36; A61K 9/00

(52) U.S. Cl. CPC A23L 2/39; A23L 2/56; A23L 29/212; A61K 9/0095; A61K 45/06; A61K 47/44; A61K 47/36; A23L 29/10

(57) ABSTRACT

Aspects described herein relate to improved methods for infusing food and beverage compositions with lipophilic active agents. More particularly, aspects described herein relate to improved methods for infusing food and beverage compositions with lipophilic active agents involving drying methods using dielectric energy, particularly microwave energy. In some aspects, the methods further comprise dehydration using dielectric energy under vacuum and stirring at a temperature of less than 70° C.

### CROSS-REFERENCE TO RELATED APPLICATIONS

This application claims priority to U.S. provisional application Ser. No. 62/519,511, filed Jun. 14, 2017, the entire disclosure of which is hereby fully incorporated herein by reference.

### TECHNICAL FIELD

Aspects described herein relate to improved methods for infusing food and beverage compositions with lipophilic active agents involving drying methods using dielectric energy, particularly microwave energy.

### BACKGROUND

Dehydration of organic materials is commonly used in the food processing industry and in the production of biologically-active materials. Dehydration may be used in order to preserve the products for storage. It may also be used to create a product that is used in the dehydrated form, for example dried herbs and various kinds of chips. Conventional methods of dehydrating organic products include air-drying and freeze-drying (lyophilization). Both of these drying methods have their limitations. In general terms, air-drying is slow and freeze-drying is expensive, and both methods tend to degrade the appearance and texture of the products, which is undesirable in the case of foods.

Furthermore, methods for infusing food and beverage compositions with lipophilic active agents have encountered technical difficulties relating to the poor water-solubility of the lipophilic agents. When administered in the form of an oil solution or some kind of water and/or oil suspension or emulsion, lipophilic compounds usually show poor bioavailability. Use of water-miscible organic solvents such as ethanol or propylene glycol also results in low bioavailability when the resulting solution is admixed with blood or gastrointestinal fluids. Other approaches of using lipophlic agent derivatives or analogs having greater solubility in water also involve a loss of bioactivity, while the use of water-soluble pro-drugs are not universally applicable to all lipophilic active agents and are relatively complicated and expensive.

Therefore, there is a need for methods for infusing food and beverage compositions with lipophilic active agents that involve improved drying methods while retaining the bioactivity of lipophilic active agents.

### SUMMARY

To address the foregoing problems, in whole or in part, and/or other problems that may have been observed by persons skilled in the art, the present disclosure provides compositions and methods as described by way of example as set forth below.

In one aspect, a dielectric energy dehydration process for making a lipophilic active agent infused food product is provided comprising the steps of:
(a) contacting a food product with an oil comprising a lipophilic active agent and a bioavailability enhancing agent, wherein the bioavailability enhancing agent comprises an edible oil comprising long chain fatty acids; and
(b) dehydrating the food product using dielectric energy;
   thereby producing the lipophilic active agent infused food product, wherein the bioavailability of the lipophilic active agent in a subject is at least 1.5 times greater than the bioavailability of the lipophilic active agent in the subject in the absence of the edible oil comprising long chain fatty acids. In other aspects, the dielectric energy is selected from the group consisting of radio frequency energy, low frequency (conventional) microwave energy, and high frequency (millimeter wave) microwave energy. In a further aspect, step (b) further comprises dehydrating the food product using dielectric energy under vacuum and stirring at a temperature of less than 70° C. In some aspects, the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

In a further aspect of the dielectric energy dehydration process for making a lipophilic active agent infused food product, step (a) further comprises saturating the food product in an edible oil or fat comprising the lipophilic active agent and a flavoring agent, thereby producing a lipophilic active agent infused food product further comprising a flavoring agent. In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In a further aspect of the dielectric energy dehydration process for making a lipophilic active agent infused food product, the bioavailability of the lipophilic active agent in a subject is at least 3 times greater than the bioavailability of the lipophilic active agent in the subject in the absence of the edible oil comprising long chain fatty acids, particularly at least 4.5 times greater. In other aspects, the edible oil comprising long chain fatty acids is substantially free of omega-6 fatty acids.

In another embodiment, a lipophilic active agent infused food product is provided, obtainable by the steps of any of the dielectric energy dehydration processes described above for making a lipophilic active agent infused food product.

In another embodiment, a dielectric energy dehydration process for making a lipophilic active agent infused beverage product is provided comprising making lipophilic active agent infused tea leaves, coffee beans, or cocoa powder according to steps (a) and (b); and further comprising the step of steeping the lipophilic active agent infused tea leaves, coffee beans, or cocoa powder in a liquid, thereby producing the lipophilic active agent infused beverage product.

In another embodiment, a lipophilic active agent infused beverage product is provided, obtainable by the steps of any of the dielectric energy dehydration processes described above for making a lipophilic active agent infused beverage product.

In another aspect, a dielectric energy dehydration process is provided for making a ready-to-drink beverage composition comprising a lipophilic active agent, comprising the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture using dielectric energy, thereby producing a dehydrated mixture comprising the emulsifier, the oil comprising the lipophilic active agent, and the bioavailability enhancing agent; and
(c) combining the dehydrated mixture with a ready-to-drink beverage composition;
   thereby producing the ready-to-drink beverage composition comprising the lipophilic active agent, wherein:
(i) the bioavailability enhancing agent comprises an edible oil comprising long chain fatty acids; and
(ii) the bioavailability of the lipophilic active agent in a subject is at least 1.5 times greater than the bioavailability of the lipophilic active agent in the subject in the absence of the edible oil comprising long chain fatty acids.
   In other aspects, the dielectric energy is selected from the group consisting of radio frequency energy, low frequency (conventional) microwave energy, and high frequency (millimeter wave) microwave energy. In a further aspect, step (b) further comprises dehydrating the food product using dielectric energy under vacuum and stirring at a temperature of less than 70° C. In some aspects, the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

In a further aspect of the dielectric energy dehydration process for making a ready-to-drink beverage composition comprising a lipophilic active agent, step (a) further comprises saturating the food product in an edible oil or fat comprising the lipophilic active agent and a flavoring agent, thereby producing a lipophilic active agent infused food product further comprising a flavoring agent. In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In a further aspect of the dielectric energy dehydration process for making a ready-to-drink beverage composition comprising a lipophilic active agent, the bioavailability of the lipophilic active agent in a subject is at least 3 times greater than the bioavailability of the lipophilic active agent in the subject in the absence of the edible oil comprising long chain fatty acids, particularly at least 4.5 times greater. In other aspects, the edible oil comprising long chain fatty acids is substantially free of omega-6 fatty acids.

In another embodiment, a ready-to-drink beverage composition comprising a lipophilic active agent is provided, obtainable by the steps of any of the dielectric energy dehydration processes described above for making a ready-to-drink beverage composition comprising a lipophilic active agent.

In further aspects, systems are provided for performing any of the dielectric energy dehydration processes described above, including hardware components and/or software components configured to perform any of the dielectric energy dehydration processes described above. In one aspect, the present invention also provides a computer readable medium programmed to cause one or more hardware components to perform any of the steps of any of the dielectric energy dehydration processes described above. In another aspect, the present invention also provides a system comprising one or more hardware components coupled to and controlled by a computer programmed to cause one or more hardware components to perform any of the steps of any of the dielectric energy dehydration processes described above.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### DETAILED DESCRIPTION

The presently disclosed subject matter now will be described more fully hereinafter. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Aspects described herein relate to improved methods for infusing food and beverage compositions with lipophilic active agents. More particularly, aspects described herein relate to the surprising discovery that dielectric energy such as radio frequency energy, low frequency (conventional) microwave energy, and high frequency (millimeter wave) microwave energy can be used to significantly improve dehydration steps within methods for infusing food and beverage compositions with lipophilic active agents while retaining a high level of bioavailability of the lipophilic active agents.

### Dielectric Energy Dehydration Processes, Compositions, and Systems

### Bioavailability

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Bioavailability is usually assessed by determining the area under the plasma concentration-time curve (AUC). AUC is directly proportional to the total amount of unchanged drug that reaches systemic circulation. Plasma drug concentration increases with extent of absorption; the maximum (peak) plasma concentration is reached when drug elimination rate equals absorption rate. Peak time is the most widely used general index of absorption rate; the slower the absorption, the later the peak time.

The bioavailability of some drugs is increased when co-administered with food, particularly agents such as cannabinoids that are Class II drugs under the Biopharmaceutical Drug Classification System (Kelepu et al. (2013) Acta Pharmaceutica Sinica B 3:361-372; Amidon et al. (1995) Pharm. Res. 12:413-420; Charman et al. (1997) J. Pharm. Sci. 86:269-282; Winstanley et al. (1989) Br. J. Clin. Pharmacol. 28:621-628). It is the lipid component of the food that plays a key role in the absorption of lipophilic drugs and that leads to enhanced oral bioavailability (Hunt & Knox (1968) J. Physiol. 194:327-336; Kelepu et al. (2013) Acta Pharmaceutica Sinica B 3:361-372). This has been attributed to the ability of a high fat meal to stimulate biliary and pancreatic secretions, to decrease metabolism and efflux activity, to increase intestinal wall permeability, and to a prolongation of gastrointestinal tract (GIT) residence time and transport via the lymphatic system (Wagnera et al. (2001) Adv. Drug Del. Rev. 50:S13-31; Kelepu et al. (2013) Acta Pharmaceutica Sinica B 3:361-372). High fat meals also elevate triglyceride-rich lipoproteins that associate with drug molecules and enhance intestinal lymphatic transport, which leads to changes in drug disposition and changes the kinetics of the pharmacological actions of poorly soluble drugs (Gershkovich et al. (2007) Eur. J. Pharm. Sci. 32:24-32; Kelepu et al. (2013) Acta Pharmaceutica Sinica B 3:361-372). However, co-administration of food with lipophilic drugs requires close control and/or monitoring of food intake when dosing such drugs, and can also be subject to problems with patient compliance (Kelepu et al. (2013) Acta Pharmaceutica Sinica B 3:361-372).

As described above, the present invention relates, in part, to the surprising discovery that dielectric energy such as radio frequency energy, low frequency (conventional) microwave energy, and high frequency (millimeter wave) microwave energy can be used to significantly improve dehydration steps within methods for infusing food and beverage compositions with lipophilic active agents while retaining a high level of bioavailability of the lipophilic active agents.

### Dielectric Energy Dehydration Processes and Compositions

In one aspect, a dielectric energy dehydration process for making a lipophilic active agent infused food product is provided comprising the steps of:
(a) contacting a food product with an oil comprising a lipophilic active agent and a bioavailability enhancing agent, wherein the bioavailability enhancing agent comprises an edible oil comprising long chain fatty acids; and
(b) dehydrating the food product using dielectric energy;
   thereby producing the lipophilic active agent infused food product, wherein the bioavailability of the lipophilic active agent in a subject is at least 1.5 times greater than the bioavailability of the lipophilic active agent in the subject in the absence of the edible oil comprising long chain fatty acids. In other aspects, the dielectric energy is selected from the group consisting of radio frequency energy, low frequency (conventional) microwave energy, and high frequency (millimeter wave) microwave energy. In a further aspect, step (b) further comprises dehydrating the food product using dielectric energy under vacuum and stirring at a temperature of less than 70° C. In some aspects, the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

In a further aspect of the dielectric energy dehydration process for making a lipophilic active agent infused food product, step (a) further comprises saturating the food product in an edible oil or fat comprising the lipophilic active agent and a flavoring agent, thereby producing a lipophilic active agent infused food product further comprising a flavoring agent. In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In a further aspect of the dielectric energy dehydration process for making a lipophilic active agent infused food product, the bioavailability of the lipophilic active agent in a subject is at least 3 times greater than the bioavailability of the lipophilic active agent in the subject in the absence of the edible oil comprising long chain fatty acids, particularly at least 4.5 times greater. In other aspects, the edible oil comprising long chain fatty acids is substantially free of omega-6 fatty acids.

In another embodiment, a lipophilic active agent infused food product is provided, obtainable by the steps of any of the dielectric energy dehydration processes described above for making a lipophilic active agent infused food product.

In another embodiment, a dielectric energy dehydration process for making a lipophilic active agent infused beverage product is provided comprising making lipophilic active agent infused tea leaves, coffee beans, or cocoa powder according to steps (a) and (b); and further comprising the step of steeping the lipophilic active agent infused tea leaves, coffee beans, or cocoa powder in a liquid, thereby producing the lipophilic active agent infused beverage product.

In another embodiment, a lipophilic active agent infused beverage product is provided, obtainable by the steps of any of the dielectric energy dehydration processes described above for making a lipophilic active agent infused beverage product.

In another aspect, a dielectric energy dehydration process is provided for making a ready-to-drink beverage composition comprising a lipophilic active agent, comprising the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture using dielectric energy, thereby producing a dehydrated mixture comprising the emulsifier, the oil comprising the lipophilic active agent, and the bioavailability enhancing agent; and
(c) combining the dehydrated mixture with a ready-to-drink beverage composition;
   thereby producing the ready-to-drink beverage composition comprising the lipophilic active agent, wherein:
(i) the bioavailability enhancing agent comprises an edible oil comprising long chain fatty acids; and
(ii) the bioavailability of the lipophilic active agent in a subject is at least 1.5 times greater than the bioavailability of the lipophilic active agent in the subject in the absence of the edible oil comprising long chain fatty acids.
   In other aspects, the dielectric energy is selected from the group consisting of radio frequency energy, low frequency (conventional) microwave energy, and high frequency (millimeter wave) microwave energy. In a further aspect, step (b) further comprises dehydrating the food product using dielectric energy under vacuum and stirring at a temperature of less than 70° C. In some aspects, the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

In a further aspect of the dielectric energy dehydration process for making a ready-to-drink beverage composition comprising a lipophilic active agent, step (a) further comprises saturating the food product in an edible oil or fat comprising the lipophilic active agent and a flavoring agent, thereby producing a lipophilic active agent infused food product further comprising a flavoring agent. In some aspects, step (b) further comprises contacting the food product with a starch, particularly wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In a further aspect of the dielectric energy dehydration process for making a ready-to-drink beverage composition comprising a lipophilic active agent, the bioavailability of the lipophilic active agent in a subject is at least 3 times greater than the bioavailability of the lipophilic active agent in the subject in the absence of the edible oil comprising long chain fatty acids, particularly at least 4.5 times greater. In other aspects, the edible oil comprising long chain fatty acids is substantially free of omega-6 fatty acids.

In another embodiment, a ready-to-drink beverage composition comprising a lipophilic active agent is provided, obtainable by the steps of any of the dielectric energy dehydration processes described above for making a ready-to-drink beverage composition comprising a lipophilic active agent.

In some aspects, vacuum drying may also be combined with dielectric energy drying. Dielectric energy vacuum drying (e.g., microwave vacuum drying) is the result of combining two technologies: 1) dielectric energy drying, which provides a fast and uniform drying and allowing that the heat is uniformly generated inside the solid; and 2) the vacuum, which allows reducing the drying temperatures (such as less than 70° C.). Microwave vacuum drying is generally used in the food processing industry, for example, for dehydrating fruits and vegetables (e.g., PCT Patent Application Pub. No. WO2009/066259 and US Patent Application Pub. No. US2008/0179318) and in the pharmaceutical industry, for example, for drying and/or granulating active ingredients (Berteli et al. (2009) Braz. J. Chem. Eng. 26:317-329; Farrel (2005) Drying Technology 23:2131-2146; McLoughlin (2003) Drying Technology 21:1719-1733) or for dehydrating temperature-sensitive biological materials (e.g., PCT Patent Application Pub. No. WO2010/124375).

In a further aspect, where the disclosed processes provide for lipophilic active agent infused food products, the processes may also include the step of lyophilizing the lipophilic active agent infused food product. Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use. Tang et al. (2004) Pharmaceutical Research 21:191-200 describes the scientific principles pertaining to freeze drying and guidelines for designing suitable freeze drying processes. Further description of freeze drying is found in Remington (2006) The Science and Practice of Pharmacy, 21^{st} edition, Lippincott Williams & Wilkins, pp. 828-831.

In a further aspect, where the disclosed processes provide for lipophilic active agent infused tea leaves, coffee beans, or cocoa powder, the process further comprises packaging the lipophilic active agent infused tea leaves, coffee beans, or cocoa powder in single or multiple serve delivery devices, such as tea bags, water permeable membranes, pre-packaged beverage pods such as K-CUP® packs manufactured and sold by Keurig Inc. of Wakefield, Mass., and the like. Examples include, but are not limited to, such delivery devices and related systems as described in U.S. Pat. Nos. 3,450,024; 5,325,765; 5,840,189; and 6,606,938.

In other aspects, the bioavailability enhancing agent within the compositions and methods of the present invention, the lipophilic active agent is an edible oil or fat, a protective colloid, or both a protective colloid and an edible oil or fat. In another aspect, the bioavailability enhancing agent is also a lipophilic active agent taste masking agent. In another particular aspect, where the bioavailability enhancing agent is both a protective colloid, an edible oil or fat, and a lipophilic active agent taste masking agent, the bioavailability enhancing agent is nonfat dry milk. In a further aspect, the bioavailability enhancing agent is substantially free of omega-6 fatty acids. In other aspects, the bioavailability of the lipophilic active agent in a subject is at least about 1.5 times, 2 times, 5 times, or 10 times greater than the bioavailability of the lipophilic active agent in the subject in the absence of the bioavailability enhancing agent. In a further aspect, the bioavailability of the lipophilic active agent in a subject is greater than 20%.

An edible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Some examples of digestible oils for use in this invention thus include: vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (such as fish liver oil, shark oil and mink oil).

Examples of protective colloids include polypeptides (such as gelatin, casein, and caseinate), polysaccharides (such as starch, dextrin, dextran, pectin, and gum arabic), as well as whole milk, skimmed milk, milk powder or mixtures of these. However, it is also possible to use polyvinyl alcohol, vinyl polymers, for example polyvinylpyrrolidone, (meth)acrylic acid polymers and copolymers, methylcellulose, carboxymethylcellulose, hydroxypropylcellulose and alginates. For further details, reference may be made to R. A. Morton, Fast Soluble Vitamins, Intern. Encyclopedia of Food and Nutrition, Vol. 9, Pergamon Press 1970, pages 128-131.

Oral administration constitutes the preferred route of administration for a majority of drugs. However, drugs that have an undesirable or bitter taste leads to lack of patient compliance in the case of orally administered dosage forms. In such cases, taste masking is an essential tool to improve patient compliance. Because lipophilic active agents (e.g., cannabinoids such as cannabidiol) have an undesirable taste profile, in order to improve compliance, the presently disclosed compositions also comprise one or more lipophilic active agent taste masking agents. Examples of lipophilic active agent taste-masking agents include dry milk as described above, as well as menthol, sweeteners, sodium bicarbonate, ion-exchange resins, cyclodextrin inclusion compounds, adsorbates, and the like.

In a further aspect, the bioavailability enhancing agent is substantially free of omega-6 fatty acids. As used herein, “substantially free” means largely but not wholly pure.

In other aspects, the bioavailability of the lipophilic active agent in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, or 10 times greater than the bioavailability of the lipophilic active agent in the subject in the absence of the bioavailability enhancing agent.

In a further aspect, the bioavailability of the lipophilic active agent in a subject is greater than 20% or at least about 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or greater.

Assays and methods for measuring lipophilic active agent bioavailability are well known in the art (see, e.g., Rocci & Jusko (1983) Comput. Programs Biomed. 16:203-215; Shargel & Yu (1999) Applied biopharmaceutics & pharmacokinetics (4th ed.). New York: McGraw-Hill; Hu & Li (2011) Oral Bioavailability: Basic Principles, Advanced Concepts, and Applications, John Wiley & Sons Ltd.; Karschner et al. (2011) Clinical Chemistry 57:66-75; Ohlsson et al. (1980) Clin. Pharmacol. Ther. 28:409-416; Ohlsson et al. (1982) Biomed. Environ. Mass Spectrom. 9:6-10; Ohlsson et al. (1986) Biomed. Environ. Mass Spectrom. 13:77-83; Karschner et al. (2010) Anal. Bioanal. Chem. 397:603-611).

### Dielectric Energy Dehydration Systems

In further aspects, systems are provided for performing any of the dielectric energy dehydration processes described above, including hardware components and/or software components configured to perform any of the dielectric energy dehydration processes described above. In particular aspects, a computer readable medium is provided wherein the computer readable medium is programmed to perform any of the dielectric energy dehydration processes described above. A system comprising hardware components coupled to and controlled by a computer programmed to cause the hardware components to perform any of the dielectric energy dehydration processes described above.

Accordingly, it will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Computer program code for carrying out operations of the invention may be written in an object oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network may even include wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

Accordingly, in one aspect, the present invention also provides a computer readable medium programmed to cause one or more hardware components to perform any of the steps of any of the processes disclosed herein.

In another aspect, the present invention also provides a system comprising one or more hardware components coupled to and controlled by a computer programmed to cause one or more hardware components to perform any of the steps of any of the processes disclosed herein.

In another aspect, it is important to be able to regulate the power setting of microwave heating over time because microwave processing at any given power level, if kept constant, results in a continual increase in heat rather than a stable heat level as one would produce in a convection oven.

Control of a batch microwave dehydration process and/or vacuum dehydration process requires consideration of several factors. For example, microwaves are most often absorbed better by the solvent (e.g., water) of the food product, mixture, or the like than the food product, mixture, or the like itself. The amount of energy absorbed by the combination of the solvent and the food product, mixture, or the like therefore decreases as the percentage of solvent within the product decreases. During vaporization of the solvent, the temperature of combination of the solvent and the food product, mixture, or the like will remain relatively stable, since energy absorbed by the solvent will be used for the vaporization. However, as vaporization nears completion, the temperature of the combination of the solvent and the food product, mixture, or the like will begin a more rapid temperature change if the microwave field is not changed to compensate.

Accordingly, in another embodiment, a system or mechanism for batch drying of a food product, mixture, or the like with a microwave system and/or microwave vacuum system is disclosed along with a method of controlling the drying process. The system or mechanism includes a chamber for drying the food product, mixture, or the like. The food product, mixture, or the like may be contained in an internal product container. The container and chamber may be coordinated to allow easy opening on a rail system. The system or mechanism includes a means for inducing a vacuum in the chamber that can also with draw water or other solvent vapor from the chamber. A source of microwaves for the chamber is provided, and the product container may also comprise an agitation mechanism for the product, mixture, or the like. A means for controlling the drying operation is also provided. A method of controlling the dehydration process is also provided that allows for the selective halting of the drying operation upon achieving selectable parameters. The control systems also allow selectable parameters to be maintained during the drying operation. In some embodiments of the invention, selectable parameters include, but are not limited to: elapsed drying time, chamber pressure, product temperature, chamber field strength, and reflected microwave power.

Control of the dehydration process may be accomplished through at least two systems; a control unit and one or more sensors. The one or more sensors are configured to measure any of the selectable parameters described above. The control unit may comprise a series of controls and indicators. Control of the process of dehydration of the food product, mixture, or the like may be accomplished either through microcomputer operation of automatic cycles, or through manual operation of the system and human monitoring of the indicators. Thus, the controls include manual controls and automatic cycle controls. All automatic cycle controls may be operated by a microcomputer.

Pressure within the drying chamber may be maintained, for example, by operation of a valve system. The entire system may be maintained in a vacuum during a drying operation. Vacuum may be maintained at the desired pressure by opening and closing one or more valves as necessary, and a vacuum pump may operate continuously throughout the drying operation. Control of the system may be accomplished by electrical equipment contained within the control unit.

In other aspects, a combination of convection and microwave dehydration may be used to dehydrate the food product, mixture, or the like. Microwave assisted drying is accomplished by simultaneously using forced-air convective heating and drying to the surface of the product while at the same time exposing the product to microwave heating that forces the moisture that remains in the product to the surface whereby the convective heating and drying continues to dry the product. The power of the microwave may be adjusted dependent on the product, mixture, or the like to be dried as well as the desired final product moisture. As an example, the product, mixture, or the like can be conveyed through an oven that contains a tunnel that is equipped with wave-guides to feed the microwave energy to the product and chokes designed to prevent the microwaves from leaving the oven. As the product is conveyed through the tunnel the convective and microwave heating simultaneously work to lower the moisture content of the product thereby drying. Typically, the air temperature is maintained within a selected range, and the microwave power is varied dependent on the product, the time the product is in the oven, and the final moisture content desired.

### Methods of Treatment

In a further aspect, a method of treating a condition is provided, comprising administering any of the compositions disclosed herein to a subject in need thereof.

In one aspect, where the lipophilic active agent within the compositions and methods of the invention is a cannabinoid, the condition is selected from the group consisting of cardiac diseases such as heart disease, ischemic infarcts, and cardiometabolic disorders; neurological diseases such as Alzheimer's disease, Parkinson's disease, schizophrenia, and Human Immunodeficiency Virus (HIV) dementia; obesity; metabolic disorders such as insulin related deficiencies and lipid profiles, hepatic diseases, diabetes, and appetite disorders; cancer chemotherapy; benign prostatic hypertrophy; irritable bowel syndrome; biliary diseases; ovarian disorders; marijuana abuse; and alcohol, opioid, nicotine, or cocaine addiction.

In another aspect, where the lipophilic active agent within the compositions and methods of the invention is nicotine, the condition is a nicotine-related disorder such as tobacco dependence/addiction, Parkinson's disease, ulcerative colitis, Alzheimer's disease, schizophrenia, Attention Deficit Hyperactivity Disorder (ADHD), Tourette's syndrome, ulcerous colitis, and post-smoking-cessation weight control.

In another aspect, where the lipophilic active agent within the compositions and methods of the invention is an NSAID as described herein, the condition is pain, fever, and/or an inflammatory-related disease or disorder, including but not limited to asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

In another aspect, where the lipophilic active agent within the compositions and methods of the invention is a vitamin, the condition is a vitamin deficiency or condition associated with the lipophilic vitamin. In a particular aspect, where the vitamin is vitamin E as described herein, the condition is vitamin E deficiency and/or a vitamin E related disease or disorder such as ataxia associated with vitamin E deficiency.

In a further aspect, a method of enhancing the bioavailability of a lipophilic active agent is provided, comprising heating any of the compositions disclosed herein to a temperature that is greater than or equal to human body temperature. In some aspects, oral administration of any of the compositions disclosed herein to a subject in need thereof results in a heating of the compositions to a temperature that is equal to human body temperature.

In another aspect, a method of administering any of the lipophilic active agents described herein to a subject is provided, comprising oral administration of any of the compositions of the present invention. Such administration may be for any purpose, including overall health and wellness, mental acuity, alertness, recreation, and the like. As used herein, the term “subject” treated by the presently disclosed methods in their many aspects is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or the prophylactic diagnosis or treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some aspects, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

### Lipophilic Active Agents

The active agents of the present invention are effective over a wide dosage range. For example, in treating adult humans, compositions and methods of the present invention comprise dosages of lipophilic active agents from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods of the present invention comprise dosages of lipophilic active agents of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

### Cannabinoids

Cannabis sativa L. is one of the most widely used plants for both recreational and medicinal purposes. Over 500 natural constituents have been isolated and identified from C. sativa covering several chemical classes (Ahmed et al. (2008) J. Nat. Prod. 71:536-542; Ahmed et al. (2008) Tetrahedron Lett. 49:6050-6053; ElSohly & Slade (2005) Life Sci. 78:539-548; Radwan et al. (2009) J. Nat. Prod. 72:906-911; Radwan et al. (2008) Planta Medica. 74:267-272; Radwan et al. (2008) J. Nat. Prod. 69:2627-2633; Ross et al. (1995) Zagazig J. Pharm. Sci. 4:1-10; Turner et al. (1980) J. Nat. Prod. 43:169-170). Cannabinoids belong to the chemical class of terpenophenolics, of which at least 85 have been uniquely identified in cannabis (Borgelt et al. (2013) Pharmacotherapy 33:195-209).

Cannabinoids are ligands to cannabinoid receptors (CB₁, CB₂) found in the human body (Pertwee (1997) Pharmacol. Ther. 74:129-180). The cannabinoids are usually divided into the following groups: classical cannabinoids; non-classical cannabinoids; aminoalkylindole-derivatives; and eicosanoids (Pertwee (1997) Pharmacol. Ther. 74:129-180). Classical cannabinoids are those that have been isolated from C. sativa L. or their synthetic analogs. Non-classical cannabinoids are bi- or tri-cyclic analogs of tetrahydrocannabinol (THC) (without the pyran ring). Aminoalkylindoles and eicosanoids are substantially different in structure compared to classical and non-classical cannabinoids. The most common natural plant cannabinoids (phytocannabinoids) are cannabidiol (CBD), cannabigerol (CBG), cannabichromene (CBC), and cannabinol (CBN). The most psychoactive cannabinoid is Δ⁹-THC.

In recent years, marijuana and its components have been reported in scientific literature to counter the symptoms of a broad range of conditions including but not limited to multiple sclerosis and other forms of muscular spasm; movement disorders; pain, including migraine headache; glaucoma; asthma; inflammation; insomnia; and high blood pressure. There may also be utility for cannabinoids as anxiolytics, anti-convulsives, anti-depressants, anti-psychotics, anti-cancer agents, as well as appetite stimulants. Pharmacological and toxicological studies of cannabinoids have largely been focused on a synthetic analog of Δ⁹-THC (commercially available under the generic name Dronabinol). In 1985, Dronabinol was approved by the FDA for the treatment of chemotherapy associated nausea and vomiting, and later for AIDS-associated wasting and anorexia.

Therapeutic use of cannabinoids has been hampered by the psychoactive properties of some compounds (e.g., Dronabinol) as well as their low bioavailability when administered orally. Bioavailability refers to the extent and rate at which the active moiety (drug or metabolite) enters systemic circulation, thereby accessing the site of action. The low bioavailability of orally ingested cannabinoids (from about 6% to 20%; Adams & Martin (1996) Addiction 91: 1585-614; Agurell et al. (1986) Pharmacol. Rev. 38: 21-43; Grotenhermen (2003) Clin. Pharmacokinet. 42: 327-60) has been attributed to their poor dissolution properties and extensive first pass metabolism.

Cannabinoids are a heteromorphic group of chemicals which directly or indirectly activate the body's cannabinoid receptors. There are three main types of cannabinoids: herbal cannabinoids that occur uniquely in the cannabis plant, synthetic cannabinoids that are manufactured, and endogenous cannabinoids that are produced in vivo. Herbal cannabinoids are nearly insoluble in water but soluble in lipids, alcohol, and non-polar organic solvents. These natural cannabinoids are concentrated in a viscous resin that is produced in glandular structures known as trichomes. In addition to cannabinoids, the resin is rich in terpenes, which are largely responsible for the odor of the cannabis plant.

The identification of Δ⁹-tetrahydrocannabinol (THC) as a major psychoactive drug and its chemical synthesis in 1964 opened a new era of synthetic cannabinoids as pharmacological agents. Cannabinoid research has increased tremendously in recent years since the discovery of cannabinoid receptors and the endogenous ligands for these receptors. The receptors include CB1, predominantly expressed in the brain, and CB2, primarily found on the cells of the immune system. Cannabinoid receptors belong to a superfamily of G-protein-coupled receptors. They are single polypeptides with seven transmembrane α-helices, and have an extracellular, glycosylated N-terminus and intracellular C-terminus. Both CB1 and CB2 cannabinoid receptors are linked to G1/0-proteins. In addition to these receptors, endogenous ligands for these receptors capable of mimicking the pharmacological actions of THC have also been discovered. Such ligands were designated endocannabinoids and included anandamide and 2-arachidonoyl glycerol (2-AG). Anandamide is produced in the brain and peripheral immune tissues such as the spleen.

Unlike THC, which exerts its action by binding to CB1 and CB2, cannabidiol does not bind to these receptors and hence has no psychotropic activity. Instead, cannabidiol indirectly stimulates endogenous cannabinoid signaling by suppressing the enzyme that breaks down anandamide (fatty acid amide hydroxylase, “FAAH”). Cannabidiol also stimulates the release of 2-AG. Cannabidiol has been reported to have immunomodulating and anti-inflammatory properties, to exhibit anticonvulsive, anti-anxiety, and antipsychotic activity, and to function as an efficient neuroprotective antioxidant.

Cannabinoids in cannabis are often inhaled via smoking, but may also be ingested. Smoked or inhaled cannabinoids have reported bioavailabilities ranging from 2-56%, with an average of about 30% (Huestis (2007) Chem. Biodivers. 4:1770-1804; McGilveray (2005) Pain Res. Manag. 10 Suppl. A:15A-22A). This variability is mainly due to differences in smoking dynamics. Cannabinoids that are absorbed through the mucous membranes in the mouth (buccomucosal application) have bioavailabilities of around 13% (Karschner et al. (2011) Clin. Chem. 57:66-75). By contrast, when cannabinoids are ingested, bioavailability is typically reduced to about 6% (Karschner et al. (2011) Clin. Chem. 57:66-75).

Accordingly, in other aspects, within the compositions and methods of the present invention, the lipophilic active agent is a cannabinoid.

In particular aspects, at least one cannabinoid within the compositions and methods of the present invention is selected from the group consisting of:

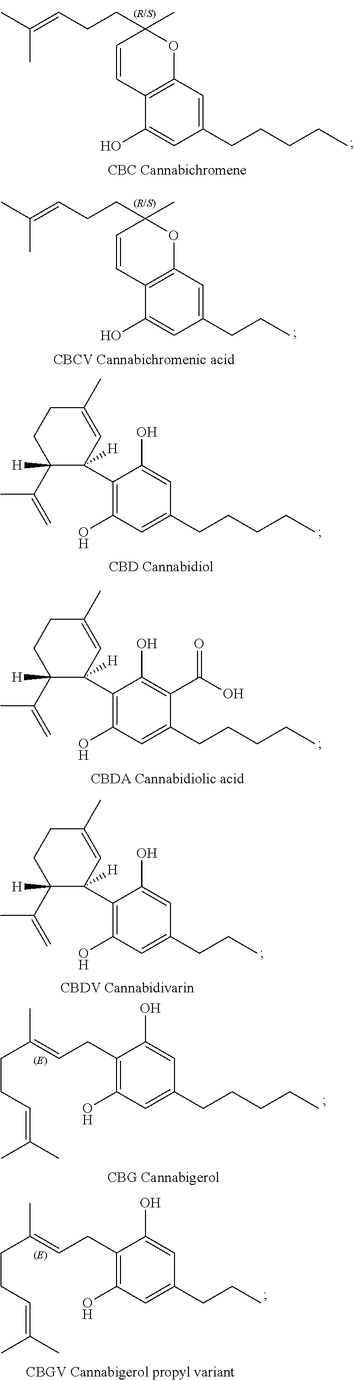

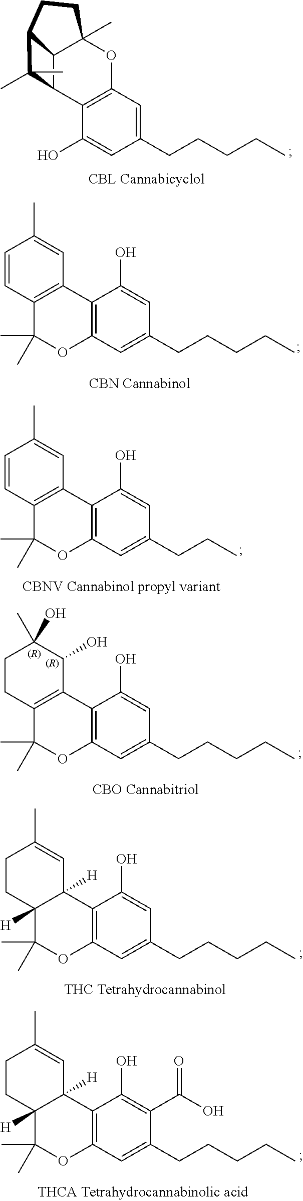

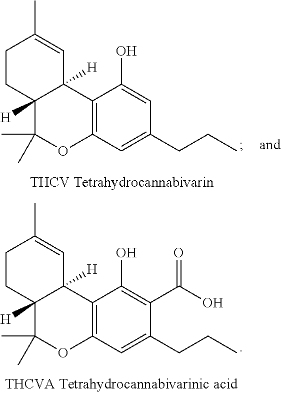

In particular aspects, at least one cannabinoid within the compositions and methods of the present invention is a non-psychoactive cannabinoid such as cannabidiol. In some particularly disclosed aspects, the cannabinoid is selected from the group consisting of:

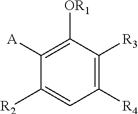

where A is aryl, and particularly

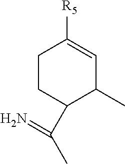

but not a pinene such as:

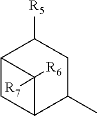

and the R₁-R₅ groups are each independently selected from the groups of hydrogen, lower substituted or unsubstituted alkyl, substituted or unsubstituted carboxyl, substituted or unsubstituted alkoxy, substituted or unsubstituted alcohol, and substituted or unsubstituted ethers, and R₆-R₇ are H or methyl. In particular aspects, there are no nitrogens in the rings, and/or no amino substitutions on the rings.

In other aspects, the cannabinoid is selected from the group consisting of:

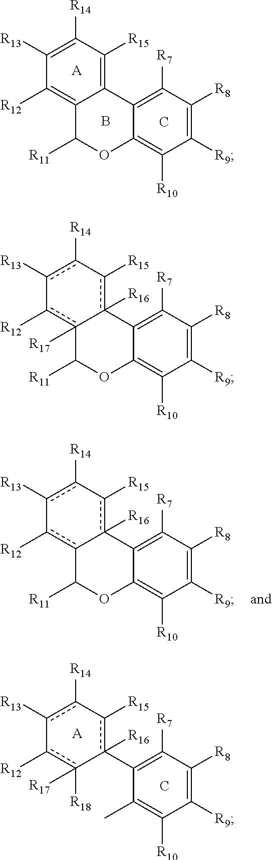

where there can be 0 to 3 double bonds on the A ring, as indicated by the optional double bonds indicated by dashed lines on the A ring. The C ring is aromatic, and the B ring can be a pyran. Particular aspects are dibenzo pyrans and cyclohexenyl benzenediols. Particular aspects of the cannabinoids of the present invention may also be highly lipid soluble, and in particular aspects can be dissolved in an aqueous solution only sparingly (for example 10 mg/ml or less). The octanol/water partition ratio at neutral pH in useful aspects is 5000 or greater, for example 6000 or greater. This high lipid solubility enhances penetration of the drug into the central nervous system (CNS), as reflected by its volume of distribution (V_{d}) of 1.5 L/kg or more, for example 3.5 L/kg, 7 L/kg, or ideally 10 L/kg or more, for example at least 20 L/kg. Particular aspects may also be highly water soluble derivatives that are able to penetrate the CNS, for example carboxyl derivatives.

R₇₋₁₈ are independently selected from the group of H, substituted or unsubstituted alkyl, especially lower alkyl, for example unsubstituted C₁-C₃ alkyl, hydroxyl, alkoxy, especially lower alkoxy such as methoxy or ethoxy, substituted or unsubstituted alcohol, and unsubstituted or substituted carboxyl, for example COOH or COCH_{3.} In other aspects R₇₋₁₈ can also be substituted or unsubstituted amino, and halogen.

In particular aspects, at least one cannabinoid within the compositions and methods of the present invention is a non-psychoactive cannabinoid, meaning that the cannabinoid has substantially no psychoactive activity mediated by the cannabinoid receptor (for example an IC₅₀ at the cannabinoid receptor of greater than or equal to 300 nM, for example greater than 1 μM and a K, greater than 250 nM, especially 500-1000 nM, for example greater than 1000 nM).

In other particular aspects, the cannabinoids within the compositions and methods of the present invention are selected from the group consisting of:

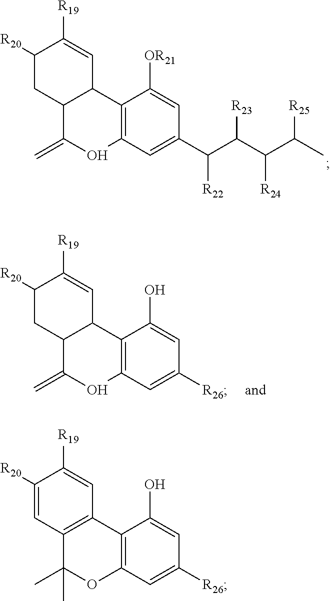

where R₁₉ is substituted or unsubstituted alkyl, such as lower alkyl (for example methyl), lower alcohol (such as methyl alcohol) or carboxyl (such as carboxylic acid) and oxygen (as in ═O); R₂₀ is hydrogen or hydroxy; R₂₁ is hydrogen, hydroxy, or methoxy; R₂₂ is hydrogen or hydroxy; R₂₃ is hydrogen or hydroxy; R₂₄ is hydrogen or hydroxy; R₂₅ is hydrogen or hydroxy; and R₂₆ is substituted or unsubstituted alkyl (for example n-methyl alkyl), substituted or unsubstituted alcohol, or substituted or unsubstituted carboxy.

In other particular aspects, the cannabinoids within the compositions and methods of the present invention are selected from the group consisting of:

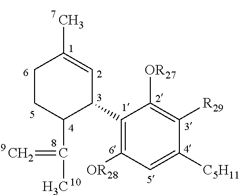

wherein numbering conventions for each of the ring positions are shown, and R₂₇, R₂₈ and R₂₉ are independently selected from the group consisting of H, unsubstituted lower alkyl such as CH₃, and carboxyl such as COCH₃. Particular examples of nonpsychoactive cannabinoids that fall within this definition are cannabidiol and

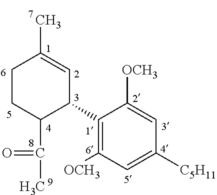

and other structural analogs of cannabidiol.

In other particular aspects, the cannabinoids within the compositions and methods of the present invention are selected from the group consisting of:

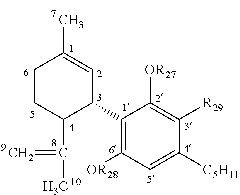

wherein R₂₇, R₂₈ and R₂₉ are independently selected from the group consisting of H, lower alkyl such as CH₃, and carboxyl such as COCH₃, and particularly wherein:
a) R₂₇═R₂₈═R₂₉═H
b) R₂₇═R₂₉═H; R₂₈═CH₃
c) R₂₇═R₂₈═CH₃; R₂₉═H
d) R₂₇═R₂₈═COCH₃; R₂₉═H
e) R₂₇═H; R₂₈═R₂₉═COCH₃
   When R₂₇═R₂₈═R₂₉═H, then the compound is cannabidiol (CBD). When R₂₇═R₂₉═H and R₂₈═CH₃, the compound is CBD monomethyl ether. When R₂₇═R₂₈═CH₃ and R₂₉═H, the compound is CBD dimethyl ether. When R₂₇═R₂₈═COCH₃ and R₂₉═H, the compound is CBD diacetate. When R₂₇═H and R₂₈═R₂₉═COCH₃, the compound is CBD monoacetate.

### Terpenes and Terpenoids

Terpenes are a diverse group of organic hydrocarbons derived from 5-carbon isoprene units and are produced by a wide variety of plants. Terpenoids are terpenes which have been chemically modified to add functional groups including heteroatoms. Terpenes and terpenoids are important building blocks for hormones, vitamins, pigments, steroids, resins, and essential oils. Terpenes are naturally present in cannabis; however, they can be removed during the extraction process. Terpenes and terpenoids have various pharmaceutical (pharmacodynamic) effects and can be selected for the desired pharmaceutical activities.

In one embodiment, the terpene/terpenoid includes limonene. Limonene is a colorless liquid hydrocarbon classified as a cyclic terpene. The more common D-isomer possesses a strong smell of oranges and a bitter taste. It is used in chemical synthesis as a precursor to carvone and as a solvent in cleaning products. Limonene is a chiral molecule. Biological sources produce one enantiomer—the principal industrial source—citrus fruit, contains D-limonene ((+)-limonene), which is the (R)-enantiomer (CAS number 5989-27-5, EINECS number 227-813-5). Racemic limonene is known as dipentene. Its IUPAC name is 1-methyl-4-(1-methylethenyl)-cyclohexene. It is also known as 4-isopropenyl-1-methylcyclohexenep-Menth-1,8-dieneRacemic: DL-limonene; dipentene.

Limonene has a history of use in medicine, food and perfume. It has very low toxicity, and humans are rarely allergic to it. Limonene is used as a treatment for gastric reflux and as an anti-fungal agent. Its ability to permeate proteins makes it a useful treatment for toenail fungus. Limonene is also used for treating depression and anxiety. Limonene is reported to assist in the absorption of other terpenoids and chemicals through the skin, mucous membranes and digestive tract. Limonene has immunostimulant properties. Limonene is also used as botanical insecticide

The principle metabolites of limonene are (+)- and (−)-trans-carveol, a product of 6-hydroxylation) and (+)- and (−)-perillyl alcohol, a product of 7-hydroxylation by CYP2C9 and CYP2C19 cytochromes in human liver microsomes. The enantiomers of perillyl alcohol have been researched for possible pharmacological possibilities as dietary chemotherapeutic agents. They are considered novel therapeutic options in some CNS neoplasms and other solid tumors, especially for treatment of gliomas. The cytotoxic activities of perillyl alcohol and limonene metabolites are likely due to their antiangiogenic properties, hyperthermia inducing effects, negative apoptosis regulation and effect on Ras pathways.

In another embodiment, the terpene/terpenoid includes linalool. Linalool is a naturally occurring terpene alcohol chemical found in many flowers and spice plants with many commercial applications, the majority of which are based on its pleasant scent (floral and slightly spicy). It is also known as β-linalool, linalyl alcohol, linaloyl oxide, p-linalool, allo-ocimenol, and 3,7-dimethyl-1,6-octadien-3-ol. Its IUPAC name is 3,7-dimethylocta-1,6-dien-3-ol.

More than 200 species of plants produce linalool, mainly in the families Lamiaceae, Lauraceae and Rutaceae. It has also been found in some fungi. Linalool has been used for thousands of years as a sleep aid. Linalool is an important precursor in the formation of Vitamin E. It has a history of use in the treatment of both psychosis and anxiety, and as an anti-epileptic agent. It also provides analgesic pain relief. Its vapors have been shown to be an effective insecticide against fleas, fruit flies and cockroaches. Linalool is used as a scent in an estimated 60-80% of perfumed hygiene products and cleaning agents including soaps, detergents, shampoos and lotions.

In another embodiment, the terpene/terpenoid includes myrcene. Myrcene, or β-myrcene, is an olefinic natural organic compound. It is classified as a hydrocarbon, more precisely as a monoterpene. Terpenes are dimers of isoprene, and myrcene is one of the most important. Myrcene is a component of the essential oil of several plants including bay, cannabis, ylang-ylang, wild thyme, mango, parsley and hops. Myrcene is produced mainly semi-synthetically from myrcia, from which it gets its name. Myrcene is a key intermediate in the production of several fragrances. α-Myrcene is the name for the structural isomer 2-methyl-6-methylene-1,7-octadiene, which is not found in nature and is little used. Its IUPAC name is 7-methyl-3-methylene-1,6-octadiene.

Myrcene has an analgesic effect and is likely to be responsible for the medicinal properties of lemon grass tea. It has anti-inflammatory properties through Prostaglandin E2. The analgesic action can be blocked by naloxone or yohimbine in mice, which suggests mediation by alpha 2-adrenoceptor stimulated release of endogenous opioids. β-Myrcene is reported to have anti-inflammatory properties, and is used to treat spasms, sleep disorders and pain. Myrcene appears to lower resistance across the blood to brain barrier, allowing itself and many other chemicals to cross the barrier more effectively.

In another embodiment, the terpene/terpenoid includes α-Pinene. α-Pinene is one of the primary monoterpenes that is physiologically critical in both plants and animals. It is an alkene and it contains a reactive four-membered ring. α-Pinene tends to react with other chemicals, forming a variety of other terpenes including D-limonene and other compounds. α-Pinene has been used for centuries as a bronchodilator in the treatment of asthma. It is highly bioavailable with 60% human pulmonary uptake with rapid metabolism. α-Pinene is an anti-inflammatory via PGE1, and appears to be a broad-spectrum antibiotic. It acts as an acetylcholinesterase inhibitor, aiding memory. Products of α-pinene which have been identified include pinonaldehyde, norpinonaldehyde, pinic acid, pinonic acid, and pinalic acid.

Pinene is found in conifer, pine and orange. α-Pinene is a major constituent in turpentine. Its IUPAC name is (1S,5S)-2,6,6-Trimethylbicyclo[3.1.1]hept-2-ene ((−)-α-Pinene).

In another embodiment, the terpene/terpenoid includes β-Pinene. β-Pinene is one of the most abundant compounds released by trees. It is one of the two isomers of pinene, the other being α-pinene. It is a common monoterpene, and if oxidized in air, the allylic products of the pinocarveol and myrtenol family prevail. Its IUPAC name is 6,6-dimethyl-2-methylenebicyclo[3.1.1]heptane and is also known as 2(10)-Pinene; Nopinene; Pseudopinene. It is found in cumin, lemon, pine and other plants.

In another embodiment, the terpene/terpenoid includes caryophyllene, also known as β-caryophyllene. Caryophyllene is a natural bicyclic sesquiterpene that is a constituent of many essential oils, including clove, cannabis, rosemary and hops. It is usually found as a mixture with isocaryophyllene (the cis double bond isomer) and α-humulene, a ring-opened isomer. Caryophyllene is notable for having a rare cyclobutane ring. Its IUPAC name is 4,11,11-trimethyl-8-methylene-bicyclo[7.2.0]undec-4-ene.

Caryophyllene is known to be one of the compounds that contribute to the spiciness of black pepper. In a study conducted by the Swiss Federal Institute of Technology, β-caryophyllene was shown to be selective agonist of cannabinoid receptor type-2 (CB2) and to exert significant cannabimimetic, anti-inflammatory effects in mice. Anti-nociceptive, neuroprotective, anxiolytic, antidepressant and anti-alcoholic activity have been tied to caryophyllene. Because β-caryophyllene is an FDA approved food additive, it is considered the first dietary cannabinoid.

In another embodiment, the terpene/terpenoid includes citral. Citral, or 3,7-dimethyl-2,6-octadienal or lemonal, is either a pair, or a mixture of terpenoids with the molecular formula C₁₀H₁₆O. The two compounds are double bond isomers. The E-isomer is known as geranial or citral A. The Z-isomer is known as neral or citral B. Its IUPAC name is 3,7-dimethylocta-2,6-dienal. It is also known as citral, geranial, neral, geranialdehyde.

Citral is present in the oils of several plants, including lemon myrtle, lemongrass, verbena, lime, lemon and orange. Geranial has a pronounced lemon odor. Neral's lemon odor is not as intense, but sweet. Citral is primarily used in perfumery for its citrus quality. Citral is also used as a flavor and for fortifying lemon oil. It has strong antimicrobial qualities, and pheromonal effects in insects. Citral is used in the synthesis of vitamin A, ionone and methylionone.

In another embodiment, the terpene/terpenoid includes humulene. Humulene, also known as α-humulene or α-caryophyllene, is a naturally occurring monocyclic sesquiterpene (C₁₅H₂₄), which is an 11-membered ring consisting of 3 isoprene units containing three nonconjugated C═C double bonds, two of them being triply substituted and one being doubly substituted. It was first found in the essential oils of Humulus lupulus (hops). Humulene is an isomer of β-caryophyllene, and the two are often found together as a mixture in many aromatic plants.

Humulene has been shown to produce anti-inflammatory effects in mammals, which demonstrates potential for management of inflammatory diseases. It produces similar effects to dexamethasone, and was found to decrease the edema formation caused by histamine injections. Humulene produced inhibitory effects on tumor necrosis factor-α (TNFα) and interleukin-1.beta. (IL1B) generation in carrageenan-injected rats. In Chinese medicine, it is blended with β-caryophyllene and used as a remedy for inflammation.

Other exemplary terpenes and terpenoids include menthol, eucalyptol, borneol, pulegone, sabinene, terpineol, and thymol. In one embodiment, an exemplary terpene/terpenoid is eucalyptol.

### Non-Steroidal Anti-Inflammatory Drugs (NSAIDs)

NSAIDs are the second-largest category of pain management treatment options in the world. The global pain management market was estimated at $22 billion in 2011, with $5.4 billion of this market being served by NSAID's. The U.S. makes up over one-half of the global market. The opioids market (such as morphine) form the largest single pain management sector but are known to be associated with serious dependence and tolerance issues.

Although NSAIDs are generally a safe and effective treatment method for pain, they have been associated with a number of gastrointestinal problems including dyspepsia and gastric bleeding.

Delivery of NSAIDs through the compositions and methods of the present invention will provide the beneficial properties of pain relief with lessened negative gastrointestinal effects, and also deliver lower dosages of active ingredients in order to provide pain management outcomes across a variety of indications.

Accordingly, in other aspects, within the compositions and methods of the present invention, the lipophilic active agent is an NSAID, particularly wherein the NSAID is selected from the group consisting of acetylsalicylic acid, ibuprophen, acetaminophen, diclofenac, indomethacin, and piroxicam.

### Vitamins

The global vitamin and supplement market is worth $68 billion according to Euromonitor. The category is both broad and deep, comprised of many popular and some lesser known substances. Vitamins in general are thought to be an $8.5 billion annual market in the U.S. The U.S. is the largest single national market in the world, and China and Japan are the 2^{nd} and 3^{rd} largest vitamin markets.

Vitamin E is fat soluble and can be incorporated into cell membranes which can protect them from oxidative damage. Global consumption of natural source vitamin E was 10,900 metric tons in 2013 worth $611.9 million.

Delivery of fat soluble vitamins through the compositions and methods of the present invention will result in less waste and lower dosages of administration. In addition, ingestion of pills is an unpleasant experience for many people so vitamin delivery through common food groups will vastly expand demand and use.

Accordingly, in other aspects, within the compositions and methods of the present invention, the lipophilic active agent is a vitamin, particularly wherein the vitamin is vitamin E.

### Nicotine

More than 99% of all nicotine that is consumed worldwide is delivered through smoking cigarettes. Approximately 6,000,000 deaths per year, worldwide, are attributed primarily to the delivery of nicotine through the act of smoking according to the Centers for Disease Control and Prevention, which also estimates that over $170 billion per year is spent just in the U.S. on direct medical care costs for adult smokers. In any twelve month period, 69% of U.S. adult smokers want to quit smoking and 43% of U.S. adult smokers have attempted to quit.

Worldwide, retail cigarette sales were worth $722 billion in 2013, with over 5.7 trillion cigarettes sold to more than 1 billion smokers.

The delivery of nicotine to satisfy current demand via the compositions and methods of the present invention, will alleviate the consumer demand for cigarettes. Since most of the adverse health outcomes of nicotine consumption are associated with the delivery method and only to a lesser degree to the actual ingestion of nicotine, a vast positive community health outcome can be achieved through the reduction in smoking cigarettes.

Accordingly, in other aspects, within the compositions and methods of the present invention, the lipophilic active agent is nicotine.

### Phosphodiesterase Type 5 Inhibitors

Phosphodiesterase type 5 inhibitors (PDE5 inhibitors) block the degradative action of cGMP-specific phosphodiesterase type 5 (PDE5) on cyclic GMP in the smooth muscle cells lining the blood vessels supplying the corpus cavernosum of the penis. These drugs, including vardenafil (Levitra®), sildenafil (Viagra®), and tadalafil (Cialis®), are administered orally for the treatment of erectile dysfunction and were the first effective oral treatment available for the condition.

PDE5 inhibitors have also been studied for other clinical use as well, including cardiovascular and heart diseases. For example, because PDE5 is also present in the arterial wall smooth muscle within the lungs, PDE5 inhibitors have also been explored for lung diseases such as pulmonary hypertension and cystic fibrosis. Pulmonary arterial hypertension, a disease characterized by sustained elevations of pulmonary artery pressure, which leads to an increased incidence of failure of the right ventricle of the heart, which in turn can result in the blood vessels in the lungs become overloaded with fluid. Two oral PDE5 inhibitors, sildenafil (Revatio®) and tadalafil (Adcirca®), are approved for the treatment of pulmonary arterial hypertension. PDE5 inhibitors have been found to have activity as both a corrector and potentiator of CFTR protein abnormalities in animal models of cystic fibrosis disease (Lubamba et al., Am. J. Respir. Crit. Care Med. (2008) 177:506-515, Lubamba et al., J. Cystic Fibrosis (2012) 11:266-273). Sildenafil has also been studied as a potential anti-inflammatory treatment for cystic fibrosis. Oral PDE5 inhibitors have also been reported to have anti-remodeling properties and to improve cardiac inotropism, independent of afterload changes, with a good safety profile (Giannetta et al., BMC Medicine (2014) 12:185). However, oral administration of PDE5 inhibitors results in poor and variable bioavailability and also extensive metabolism in the liver (Sandqvist et al., Eur. J. Clin. Pharmacol. (2013) 69:197-207; Mehrotra, Intl. J. Impotence Res. (2007) 19:253-264). If oral doses are increased beyond certain levels, the incidence of systemic side effects increase which prevents the acceptable use of these drugs. (Levitra EMEA Scientific Discussion Document, 2005).

Accordingly, in other aspects, within the compositions and methods of the present invention, the PDE5 inhibitor may include, but is not limited to, avanafil, lodenafil, mirodenafil, sildenafil (or analogs thereof, for example, actetildenafil, hydroxyacetildenafil, or dimethyl-sildenafil), tadalafil, vardenafil, udenafil, acetildenafil, or thiome-thisosildenafil. The structures of these compounds are respectively shown below:

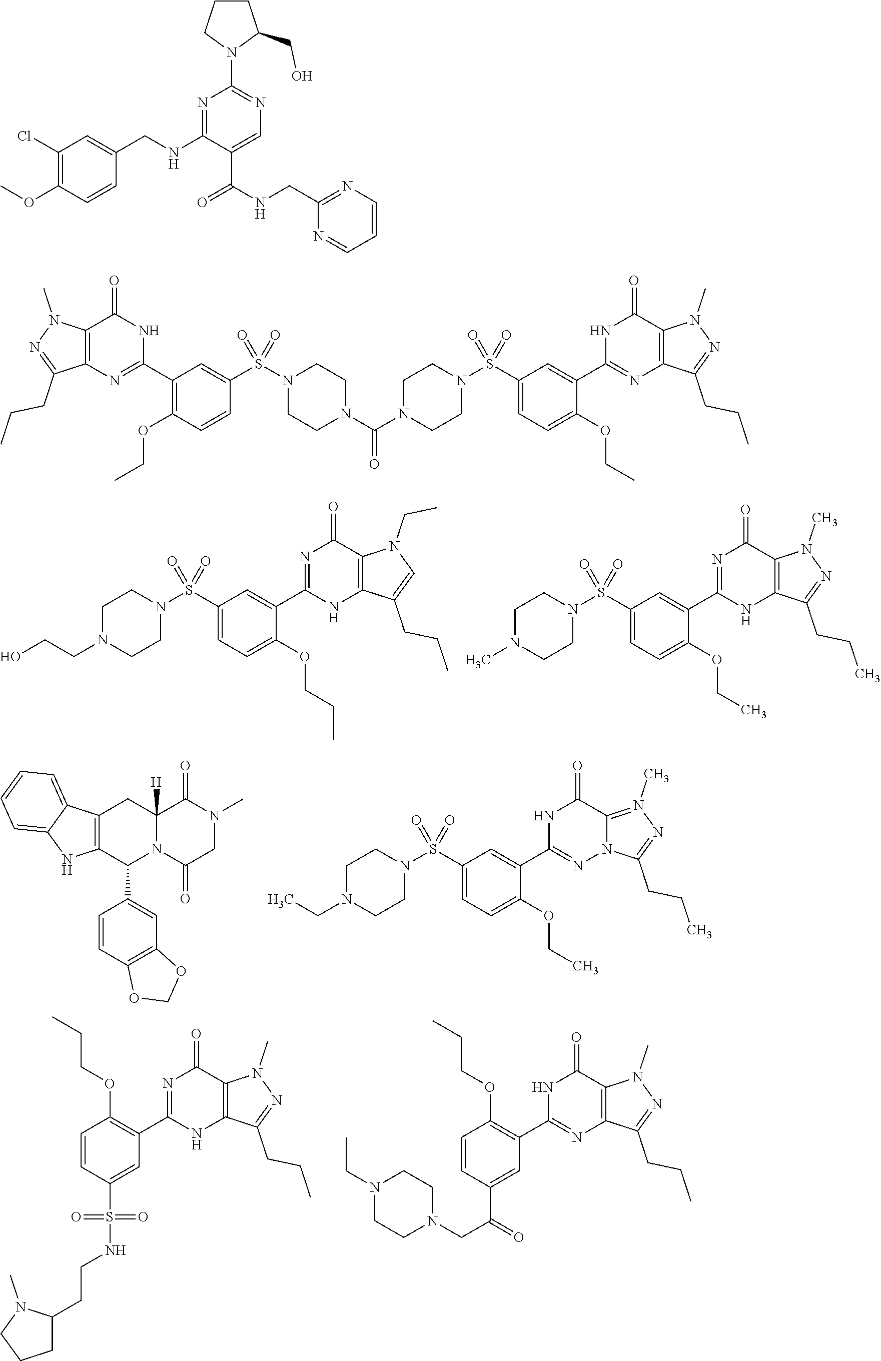

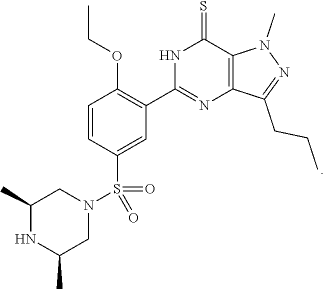 [SMILES: CCCc1nn(C)c2c(=S)[nH]c(-c3cc(S(=O)(=O)N4C[C@@H](C)N[C@@H](C)C4)ccc3OCC)nc12]

### Maca Extract

Lepidium meyenii (Maca, maca-maca, maino, ayak chichira, and ayak willku) is a Peruvian plant of the Brassicaceae family cultivated for more than 2000 years. Its main active principles are alkaloids (Macaridine, Lepidiline A and B); bencil-isotiocyanate and glucosinolates; macamides, beta-ecdysone and fitosterols. These substances activate ATP synthesis which confers energizing properties. They also diminish variations in homeostasis produced by stress because they reduce corticosterone's high levels; prevent glucose diminution and the increase of suprarenal glands' weight due to stress. They also restore homeostasis and improve energy (Lopez-Fando et al. (2004) Phytother Res. 18:471-4). A double blind placebo-controlled, randomized, parallel trial study in which active treatment with different doses of Lepidium meyenii was compared with placebo showed an improvement in sexual desire. (Gonzales et al. (2002) Andrologia 34:367-72). Lepidium meyenii also improves sperm production and sperm motility by mechanisms not related to LH, FSH, PRL, T and E2 (Gonzales et al. (2001) Asian J. Androl. 3:301-3).

### Estrogen

As used herein, “estrogen” includes estrogenic steroids such as estradiol (17-β-estradiol), estradiol benzoate, estradiol 17 β-cypionate, estropipate, equilenin, equilin, estriol, estrone, ethinyl estradiol, conjugated estrogens, esterified estrogens, and mixtures thereof.

Estrogens refer to a group of endogenous and synthetic hormones that are important for and used for tissue and bone maintenance. Estrogens are endocrine regulators in the cellular processes involved in the development and maintenance of the reproductive system. The role of estrogens in reproductive biology, the prevention of postmenopausal hot flashes, and the prevention of postmenopausal osteoporosis are well established. Estradiol is the principal endogenous human estrogen, and is found in both women and men.

The biological actions of estrogens and antiestrogens are manifest through two distinct intracellular receptors, estrogen receptor alpha (ERα) and estrogen receptor beta (ERβ). Endogenous estrogens are typically potent activators of both receptor subtypes. For example estradiol acts as an ERα agonist in many tissues, including breast, bone, cardiovascular and central nervous system tissues. Selective estrogen receptor modulators commonly act differently in different tissues. For example, a SERM may be an ERα antagonist in the breast, but may be a partial ERα agonist in the uterus, bone and cardiovascular systems. Compounds that act as estrogen receptor ligands are, therefore, useful in treating a variety of conditions and disorders.

### Progesterone and Progestins

The term “progesterone” as used herein refers to a member of the progestin family and comprises a 21 carbon steroid hormone. Progesterone is also known as D4-pregnene-3,20-dione; 4-pregnene-3,20-dione; or pregn-4-ene-3,20-dione. A progestin is a molecule whose structure is related to that of progesterone, is synthetically derived, and retains the biologically activity of progesterone. Representative synthetic progestin include, but are not limited to, modifications that produce 17a-OH esters (i.e., 17 a-hydroxyprogesterone caproate), as well as, modifications that introduce 6 a-methyl, 6-Me, 6-ene, and 6-chloro sustituents onto progesterone (i.e., medroxyprogesterone acetate, megestrol acetate, and chlomadinone acetate).

### Testosterone

Testosterone is the main androgenic hormone formed in the testes. Testosterone therapy is currently indicated for the treatment of male hypogonadism. It is also under investigation for the treatment of wasting conditions associated with AIDS and cancer, testosterone replacement in men over the age of 60, osteoporosis, combination hormone replacement therapy for women and male fertility control.

Orally administered testosterone is largely degraded in the liver, and is therefore not a viable option for hormone replacement since it does not allow testosterone to reach systemic circulation. Further, analogues of testosterone modified to reduce degradation (e.g., methyltestosterone and methandrostenolone) have been associated with abnormalities in liver function, such as elevation of liver enzymes and conjugated bilirubin. Injected testosterone produces wide peak-to-trough variations in testosterone concentrations that do not mimic the normal fluctuations of testosterone, and makes maintenance of physiological levels in the plasma difficult. Testosterone injections are also associated with mood swings and increased serum lipid levels. Injections require large needles for intramuscular delivery, which leads to diminished patient compliance due to discomfort.

To overcome these problems, transdermal delivery approaches have been developed to achieve therapeutic effects in a more patient friendly manner. For example, U.S. Pat. No. 5,460,820 discloses a testosterone-delivering patch for delivering 50 to 500 μg/day of testosterone to a woman. In addition, U.S. Pat. No. 5,152,997 discloses a device comprising a reservoir of testosterone with a skin permeation enhancer and a means for maintaining the reservoir in diffusional communication with the skin, such as an adhesive carrier device or a basal adhesive layer.

### Fentanyl

Fentanyl (also known as fentanil) is a potent synthetic narcotic analgesic with a rapid onset and short duration of action. Fentanyl is a strong agonist at μ-opioid receptors. Fentanyl is manufactured under the trade names of SUBLIMAZE, ACTIQ, DUROGESIC, DURAGESIC, FENTORA, ONSOLIS INSTANYL, ABSTRAL, and others. Historically, fentanyl has been used to treat chronic breakthrough pain and is commonly used before procedures as an anesthetic in combination with a benzodiazepine. Fentanyl is approximately 100 times more potent than morphine with 100 micrograms of fentanyl approximately equivalent to 10 mg of morphine and 75 mg of pethidine (meperidine) in analgesic activity.

Suitable analogues of fentanyl include, without limitation, the following: alfentanil (trade name ALFENTA), an ultra-short-acting (five to ten minutes) analgesic; sufentanil (trade name SUFENTA), a potent analgesic for use in specific surgeries and surgery in heavily opioid-tolerant/opioid-dependent patients; remifentanil (trade name ULTIVA), currently the shortest-acting opioid, has the benefit of rapid offset, even after prolonged infusions; carfentanil (trade name WILDNIL) an analogue of fentanyl with an analgesic potency 10,000 times that of morphine and is used in veterinary practice to immobilize certain large animals such as elephants; and lofentanil an analogue of fentanyl with a potency slightly greater than carfentanil.

### Buprenorphine

Buprenorphine (17-(cyclopropyl-methyl)-α-(1,1-dimethylethyl)-4,5-epoxy-18,19-dihy-dro-3-hydroxy-6-methoxy-α-methyl-6,14-ethenomorphinan-7-methanol) is an endoethylene morphinan derivative and a partial agonist of μ-opioid receptors with a strong analgesic effect. Buprenorphine is a partially synthetic opiate whose advantage over other compounds from this class of substance lies in a higher activity. This means that freedom from pain can be achieved in cancer or tumour patients with very unfavourable diagnosis, in the final stage, with daily doses of around 1 mg. A feature of buprenorphine in this context over the synthetic opioid fentanyl and its analogues is that the addictive potential of buprenorphine is lower than that of these compounds. A disadvantage is that, owing to the high molecular weight of buprenorphine, namely 467.64 daltons, it has been traditionally been difficult to effect its transdermal absorption.

### Scopolamine

Scopolamine is a so-called antiemitic, it is preferably used to avoid nausea and vomiting, for example, arising from repeated passive changes in the balance occurring during traveling. Scopolamine is represented by the following chemical structure:

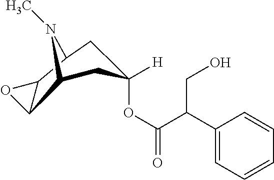

Scopolamine analogs are also encompassed by the compositions and methods of the present invention. It is understood that the phrase “scopolamine analogs” includes compounds that generally have the same backbone as scopolamine, but where various moieties have been substituted or replaced by other substituents or moieties. Some examples of scopolamine analogs that can be used in the compositions and methods disclosed herein include, but are not limited to, salts of scopolamine with various acids, such as hydrochloric acid, hydrobromic acid, hydroiodic acid, nitric acid, phosphoric acid, sulfuric acid, and the like. In one aspect, a suitable scopolamine analog can be scopolamine hydrobromide.

Additional examples of scopolamine analogs include, but are not limited to, N-alkylated analogs of scopolamine, that is, analogs containing an alkyl substituent attached to the nitrogen atom, forming a quaternary ammonium species. By “alkyl” is meant a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. The alkyl group can also be substituted or unsubstituted.

Also included are other salts (e.g., pharmaceutically acceptable salts) of such N-alkylated scopolamine analogs.

Still further examples of scopolamine analogs include, but are not limited to, un-epoxylated analogs of scopolamine, that is, analogs where the epoxy group is removed. One example of such an analog is atropine. Like scopolamine, atropine has various salt and N-alkylated analogs. These atropine analogs are intended to be included by the phrase “scopolamine analogs.” As such, further examples of scopolamine analogs include, but are not limited to, analogs of atropine with various salts (e.g., atropine hydrobromide, atropine hydrochloride, and the like) and N-alkylated analogs of atropine (e.g., atropine methyl bromide). Also included are homatropine and its salts and N-alkylated analogs.

A list of suitable scopolamine analogs that can be used in the disclosed compositions and methods, including their commercial brand names, includes, but is not limited to, atropine, atropine hydrobromide, atropine oxide hydrochloride, atropine sulfate, belladonna, scopolamine, scopolamine hydrobromide, scopolamine methylbromide, scopolamine butylbromide, homatropine, ipratropium, tiotropium, hyoscyamine sulfate, methscopolamine, methscopolamine bromide, homatropine hydrobromide, homatropine methylbromide, hyoscyamine, hyoscyamine hydrobromide, hyoscyamine sulfate, propantheline bromide, anisotropine, anisotropine methylbromide, methantheline bromide, emepronium bromide, clindinium, clidinium bromide, hyoscine, hyoscine butylbromide, hyoscine hydrobromide, hyoscine methobromide, hyoscine methonitrite, hyoscyamine, hyoscyamine sulfate, buscapine, buscolysin, buscopan, butyiscopolamine, hyoscine N-butylbromide, N-butylscopolammonium bromide, scopolan bromide, butylscopolammonium bromide, N-butylscopolammonium chloride, hyoscine N-butylbromide, DD-234, hyoscine methiodide, hyoscine methobromide, methyiscopolamine nitrate, methylscopolammoium methylsulfate, N-methylscine methylsulfate, N-methylscopolamine bromide, N-methylscopolamine iodide, N-methylscopolamine methylchloride, N-methylscopolamine methylsulfate, N-methylscopolamine nitrate, skopyl, ulix bromide, N-methylscopolamine, N-methylscopolamine methobromide, scopolamine methylchloride, N-methylscine methylsulfate, tematropium methylsulfate, and N-isopropylatropine, including salts and derivatives thereof.

### EXAMPLES

### Example 1

A line of CBD and/or THC infused tea bags coming in a variety of flavors was developed.

I. Ingredients

Tea in leaf form, oil form, brewed form, organic and inorganic

Evaporated dry non-fat milk

CBD oil

Hemp oil or compatible oil for ingestion

Cannabis leaves, buds, oils; all strains with THC and/or CBD

II. ViPova® Formulas

II A. CBD Tea

Combine evaporated nonfat, dry milk with any and all teas, organic and inorganic

Blend CBD oil with the tea leaves

Dehydrate mixture of tea, CBD oil, and evaporated nonfat dry milk using low frequency (conventional) microwave energy

End-product is ViPova® Tea with CBD enhancement only

II B. THC/CBD Tea

Combine evaporated nonfat, dry milk with any and all teas, organic and inorganic

Blend hemp or other ingestible oil with the tea leaves

Add cannabis leaves to above mixture

Dehydrate mixture of tea, hemp or other ingestible oil, cannabis leaves, and evaporated nonfat dry milk using low frequency (conventional) microwave energy

End-product is ViPova® Tea with THC and CBD

III. ViPova® Formulas: Specifications

III A. CBD Tea

Tea: one tea bag contains 1 gram to 3 grams of tea leaves (dry weight)

Evaporated dry non-fat milk: 0.10-1.00 grams

CBD oil: 10 mgs.-25 mgs. per tea bag

III B. THC/CBD Tea

Tea: one tea bag contains 1.5-12 grams tea leaves (dry weight) per tea bag

Evaporated dry milk: 0.10-6.00 grams per tea bag

Hemp oil or other ingestible oil: 10 mgs.-25 mgs. per tea bag Cannabis leaves: 1.00-12.00 grams per tea bag

III C. Production Equipment:

Commercial grinder for tea and/or cannabis leaves

Commercial mixer

Commercial microwave dryer

Commercial tea bag filling machine

IV. Flavorings

ViPova® Teas provide a menu of flavorings for addition to tea bags or loose tea selections including, but not limited to mint, citrus, and vanilla.

### Example 2

A process for adhering CBD and/or THC to food products was developed. The food products may be selected from the group consisting of meats, fish, fruits, vegetables, dairy products, legumes, pastas, breads, grains, seeds, nuts, spices, and herbs. The process may or may not involve contacting the food product with sunflower and/or dry evaporated milk. The process involved the steps of:

1. A food product was saturated with 0-60 grams of CBD and/or THC oil or extract.

2. The food product was dehydrated using low frequency (conventional) microwave energy.

3. The food product was stored in air-tight containers.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1. A dielectric energy dehydration process for making a lipophilic active agent infused food product, comprising the steps of:
(a) contacting a food product with an oil comprising a lipophilic active agent and a bioavailability enhancing agent, wherein the bioavailability enhancing agent comprises an edible oil comprising long chain fatty acids; and
(b) dehydrating the food product using dielectric energy;
thereby producing the lipophilic active agent infused food product, wherein the bioavailability of the lipophilic active agent in a subject is at least 1.5 times greater than the bioavailability of the lipophilic active agent in the subject in the absence of the edible oil comprising long chain fatty acids.

2. The process of claim 1, wherein the dielectric energy is selected from the group consisting of radio frequency energy, low frequency microwave energy, and high frequency microwave energy.

3. The process according to claim 1, wherein step (b) further comprises dehydrating the food product using dielectric energy under vacuum and stirring at a temperature of less than 70° C.

4. The process according to claim 1, wherein the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

5. The process according to claim 1, wherein step (a) further comprises saturating the food product in an edible oil or fat comprising the lipophilic active agent and a flavoring agent, thereby producing a lipophilic active agent infused food product further comprising a flavoring agent.

6. The process according to claim 1, wherein step (b) further comprises contacting the food product with a starch.

7. The process according to claim 6, wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

8. The process according to claim 1, wherein the bioavailability of the lipophilic active agent in a subject is at least 3 times greater than the bioavailability of the lipophilic active agent in the subject in the absence of the edible oil comprising long chain fatty acids.

9. The process according to claim 1, wherein the bioavailability of the lipophilic active agent in a subject is at least 4.5 times greater than the bioavailability of the lipophilic active agent in the subject in the absence of the edible oil comprising long chain fatty acids.

10. The process according to claim 1, wherein the edible oil comprising long chain fatty acids is substantially free of omega-6 fatty acids.

11. A lipophilic active agent infused food product, obtainable by the process according to claim 1.

12. A dielectric energy dehydration process for making a lipophilic active agent infused beverage product, comprising making lipophilic active agent infused tea leaves, coffee beans, or cocoa powder according the process according to claim 1; and further comprising the step of steeping the lipophilic active agent infused tea leaves, coffee beans, or cocoa powder in a liquid, thereby producing the lipophilic active agent infused beverage product.

13. A lipophilic active agent infused beverage product, obtainable by the process of claim 12.

14. A dielectric energy dehydration process for making a ready-to-drink beverage composition comprising a lipophilic active agent, comprising the steps of:
(a) contacting an emulsifier with an oil comprising a lipophilic active agent and a bioavailability enhancing agent, thereby producing a mixture comprising the emulsifier, the oil comprising the lipophilic active agent, and the bioavailability enhancing agent;
(b) dehydrating the mixture using dielectric energy, thereby producing a dehydrated mixture comprising the emulsifier, the oil comprising the lipophilic active agent, and the bioavailability enhancing agent; and
(c) combining the dehydrated mixture with a ready-to-drink beverage composition;
thereby producing the ready-to-drink beverage composition comprising the lipophilic active agent, wherein:
(i) the bioavailability enhancing agent comprises an edible oil comprising long chain fatty acids; and
(ii) the bioavailability of the lipophilic active agent in a subject is at least 1.5 times greater than the bioavailability of the lipophilic active agent in the subject in the absence of the edible oil comprising long chain fatty acids.

15. The process of claim 14, wherein the dielectric energy is selected from the group consisting of radio frequency energy, low frequency microwave energy, and high frequency microwave energy.

16. The process according to claim 14, wherein step (b) further comprises dehydrating the food product using dielectric energy under vacuum and stirring at a temperature of less than 70° C.

17. The process according to claim 14, wherein the lipophilic active agent is selected from the group consisting of cannabinoids, terpenes and terpenoids, NSAIDs, vitamins, nicotine, phosphodiesterase type 5 (PDE5) inhibitors, Maca extract, estrogen, progestin, testosterone, buprenorphine, and scopolamine.

18. The process according to claim 14, wherein step (a) further comprises saturating the food product in an edible oil or fat comprising the lipophilic active agent and a flavoring agent, thereby producing a lipophilic active agent infused food product further comprising a flavoring agent.

19. The process according to claim 14, wherein step (b) further comprises contacting the food product with a starch.

20. The process of claim 19, wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

21. The process according to claim 14, wherein the bioavailability of the lipophilic active agent in a subject is at least 3 times greater than the bioavailability of the lipophilic active agent in the subject in the absence of the edible oil comprising long chain fatty acids.

22. The process according to claim 14, wherein the bioavailability of the lipophilic active agent in a subject is at least 4.5 times greater than the bioavailability of the lipophilic active agent in the subject in the absence of the edible oil comprising long chain fatty acids.

23. The process according to claim 14, wherein the edible oil comprising long chain fatty acids is substantially free of omega-6 fatty acids.

24. A ready-to-drink beverage composition comprising a lipophilic active agent obtainable by the process according to claim 14.

25.-26. (canceled)
